(19) United States
(12) Patent Application Publication
(10) Pub. No.: US 2025/0269259 A1
(43) Pub. Date: Aug. 28, 2025

(54) ENVIRONMENTALLY CONTROLLED MULTI-MODALITY CHAMBER, COMMUNICATION SYSTEMS AND METHODS OF USE

(71) Applicant: Visibelle Derma Institute, Inc., Bloomington, MN (US)

(72) Inventor: Steven J. Daffer, Edina, MN (US)

(21) Appl. No.: 19/059,436

(22) Filed: Feb. 21, 2025

Related U.S. Application Data
(60) Provisional application No. 63/556,946, filed on Feb. 23, 2024.

(51) Int. Cl. A63B 71/04; A63B 24/00; A63B 71/06

(52) U.S. Cl. CPC A63B 71/04; A63B 24/0062; A63B 2071/0625; A63B 2213/005; A63B 2225/64; A63B 2225/66; A63B 2225/74; A63B 2230/438; A63B 2230/505

(57) ABSTRACT

A method of controlling an environmentally controlled chamber includes providing a chamber with at least one infrared heater, a screen configured to stream content, the screen positioned behind an insulating barrier comprising a substantially optically transparent portion, a camera configured to record a user and speakers within the chamber. The method includes sending the user's physiological and/or psychological data and environmental conditions within the chamber to a location remote from the chamber for monitoring, and changing environmental conditions within the chamber based upon the received physiological and/or psychological data or sensed environmental conditions from the location remote from the chamber.

### CROSS-REFERENCE TO RELATED APPLICATION

The present application is based on and claims the benefit of U.S. provisional patent application Ser. No. 63/556,946, filed Feb. 23, 2024, the content of which is hereby incorporated by reference in its entirety.

### BACKGROUND

The present disclosure relates to a communication and control system for use with an environmentally adjustable chamber such that the environment within the chamber can be modified for one or more users within the chamber. More particularly, the communication and control system for use with at least one environmentally adjustable chamber allows an environment to be adjusted in real time while the communication system allows an observer at a location remote from the chamber to communicate with the one or more persons conducting an activity within the chamber in real time.

Many people want to exercise, train, do physical therapy and/or rehabilitation activities at sub-ambient or elevated temperatures for the enhanced results relative to conducting the same activities at ambient temperatures. Exercising, training, and/or conducting physical therapy and rehabilitation at sub-ambient or elevated temperatures has many benefits. Some non-limiting benefits include increasing the rate at which calories are consumed and increasing and decreasing body temperature and stresses on the body, both of which increase a person's performance compared to conducting the same activities at ambient conditions. Additionally, exercising, training, and/or conducting physical therapy and rehabilitation at sub-ambient or elevated temperatures can increase cardiovascular health and stamina and may allow a person to achieve a greater range of motion to increase flexibility.

There is a need to customize an environment for people conducting activities including, but not limited to, elevating temperatures above ambient temperatures and/or below ambient temperatures for one or more users. There is also a need to customize a physical activity regime for the one or more users by receiving real time feedback while performing the activity within the chamber. There is also a need for an observer to be remote from the controlled environment chamber while providing real time feedback to the user, as the observer cannot withstand prolonged times within the controlled environment chamber due to the physical demands on the body.

### SUMMARY

An aspect of the present disclosure relates a method of controlling an environmentally controlable chamber. The method includes providing a chamber with at least one infrared heater, a screen configured to stream content, the screen positioned behind an insulating barrier comprising a substantially optically transparent portion, a camera configured to record a user and speakers within the chamber. The method includes sending the user's physiological and/or psychological data and environmental conditions within the chamber to a location remote from the chamber for monitoring, and changing environmental conditions within the chamber based upon the received physiological and/or psychological data or sensed environmental conditions from the location remote from the chamber.

Another aspect of the present disclosure includes a method of controlling a plurality of environmentally controlled chambers. The method includes providing a plurality of spaced apart chamber with at least one infrared heater, a screen configured to stream content, the screen positioned behind an insulating barrier comprising a substantially optically transparent portion, a camera configured to record a user and speakers within the chamber. The method includes sending the user's physiological and/or psychological data and environmental conditions within each of the plurality of chambers to a location remote from the chamber for monitoring. The method also includes changing environmental conditions within one of the plurality of chambers based upon the received physiological and/or psychological data or sensed environmental conditions within the one chamber of the plurality of chambers based upon signals sent from the location remote from the one chamber of the plurality of chambers.

### BRIEF DESCRIPTION OF THE DRAWINGS

FIG. 1 is a cutaway perspective view of a chamber with a portion of a control system.

FIG. 1A is an enlarged view of a recess configured to retain a hand held portable electronic device.

FIG. 2 is a cutaway perspective view of the chamber with another portion of the control system.

FIG. 3 is a cutaway perspective view of the chamber with yet another portion of the control system.

FIG. 4 is a schematic view of a chamber with controllable environmental modalities.

FIG. 5 is a schematic view of an observer's equipment.

FIG. 6 is an exemplary graphic user interface for the observer's use.

FIG. 7 is a schematic view of a control system for monitoring and controlling chambers from a common facility.

FIG. 8 is a schematic view of a control system with a plurality of observers located in a separate facility from the chambers.

FIG. 9 is a schematic view of a control system for residential use.

FIG. 10 is a schematic view of a control system with an observer located in a facility remote from users in a plurality of facilities.

### DEFINITIONS

An “user” as used herein is a person that is using a chamber with a customizable environment. Non-limiting activities that the user can be doing include actively exercising for cardio vascular condition, strength training, speed and agility training or flexibility training. The user can also be passively using the chamber by sitting, standing, laying prone and/or for relaxation or meditation purposes. The user can also be using the chamber for physical rehabilitation purposes.

An “observer” as used herein is a person that is located in a position that is spaced from the chamber where the observer interacts with one or more users in one or more chambers. The observer can be a physical trainer who is working with one or more users to maximize physical benefits from exercising in a chamber, a physical therapist or other medical professional or a person that monitors the user while the user is in the chamber. The observer can monitor one or more users, the one or more user's physiological data, and/or environmental conditions within the one or more chambers. Based upon the received inputs regarding the conditions in the one or more chambers and the one or more user's physiological data and/or psychological data, the observer either adjust environmental conditions in the one or more chambers based upon the physiological data of the one or more users and/or instruct modification of the one or more user's activities. The observer can also communicate with the one or more users in the one or more chambers utilizing one-way or two-way communication.

A “control system” as used herein includes a communication system and a system for controlling and adjusting one or more environmental conditions in the one or more chambers. The control system is configured to control the environmental conditions in each of the one or more chambers while allowing for communication between an observer and a user independent from the other one or more chambers.

A “one-way” communication system within the control system as used herein is a system that allows one or more observers to view a video stream of one or more users in one or more environmentally controllable chambers where the one or more observers can select to audibly communicate with one or more selected users in one or more environmentally controllable chambers independent from the other chambers.

A “two-way” communication system within the control system as used herein is a system that allows one or more observers and the one or more users to send and receive video streams of each other and where the observer can select to audibly communicate with one or selected users in one or more chambers. An alternative two-way communication allows the user and the observer to communicate in real time while the observer receives a video stream of the user. Another two-way communication systems allows video streams of the user and observer to be viewed by the other person and allows the user and observer to communicate in real time.

A “wearable device” as used herein is a device that is worn by a user that is configured to monitor or sense physiological data of the user and to transmit the monitored or sensed physiological data to the control system.

“Physiological and/or psychological data” as used herein includes, but not limited to, internal body temperature, pulse rate, heart rate, blood pressure, blood oxygen content, eye movement, surface skin temperature, range of motion, strength output, speed of movement, speed of reaction, repetition of movement, maximum rate of oxygen consumption that the body is able to use (VO_{2 max}) and distance moved during exercise.

“Environmental conditions” as used herein includes, but is not limited to, temperature, humidity, salt and mineral content, visible light wavelength, ultraviolet wavelength, infrared wavelength, biomagnetism, pulsed electromagnetism, air speed and volumetric flow rate, oxygen content, sound, ultrasound waves, radiofrequency waves, visual content and/or holistically enhanced air and/or infused aroma in the chamber.

### DETAILED DESCRIPTION

The present disclosure relates to at least one chamber that is configured to have a customizable environment, including an elevated or sub-ambient temperature, and a one-way or two-way communication system within a control system associated with the at least one chamber. The control system allows one or more observers to view one or more users in one or more chambers and provide at least audible feedback in real time to the user and/or adjust environmental conditions within the one or more chambers from a location remote from the one or more chambers. The combination of the one or more chambers and the control system allows the environmental conditions within the one or more chamber to be monitored and adjusted, while allowing the one or more users to receive real time feedback or instruction from the one or more observers that is located remote from the one or more chamber. Additionally, the one or more observers can visually monitor the one or more users and/or the one or more user's physiological data and/or psychological data such that the results of activity in the one or more environmentally controlled chambers can be more effective.

The present disclosure also provides components of the communication system within the control system within each of the one or more chambers that allows the one or more users to conduct the selected activity, whether active or passive, while being viewed by one or more observers in real time, where the one or more observers can provide feedback in real time to the user from a location remote from the chamber. The communications system within the control system includes at least a camera that that is configured to transmit a video stream of the user within the chamber to a screen that is viewed by the observer at a location remote from the chamber in real time. The communications system within the control system also includes at least a speaker in the chamber that is configured to provide audible feedback spoken into a microphone by the observer at the location remote from the chamber and transmitted through the communication system within the control system to the user within the chamber in real time. The configuration of the camera and speaker in the chamber and the screen and microphone remote from the chamber is a one-way communication from the observer to the user, as the observer can provide feedback to the user while the user cannot communicate with the observer or trainer.

In another embodiment, the chamber includes the camera and the speaker, as well as a microphone and an optional screen. The observer also has a speaker and a camera along with the microphone and the screen. With each party having access to a camera, a speaker, a microphone and a screen, the user and the observer can see and converse with each other in real time, making the chamber and/or the exercise experience more interactive. The configuration of the control system where each party has a screen, a microphone, a video camera and a speaker is a two-way communication between the observer or trainer and the user. Another two-way communication system is a configuration where the observer and user can communicate in real time while the observer receives a video stream of the user.

In some embodiments, the screen, microphone, video camera and speaker are individual components. In other embodiments components can be combined into one or more pieces of equipment. In some embodiments, the equipment is contained within a smart television, having a camera and microphone along with the screen and speakers.

In some embodiments, the observer is located within the same space at a location of a chamber where one-way or two-way communication in the control system is utilized between the user in the chamber and the observer. In another embodiment, the observer is located within the same space at a location of a plurality of chambers wherein the control system allows the observer to communicate with one or more users in each of the plurality of chambers in real time with either the one-way communication or the two-way communication. In some embodiments, the observer can choose between the one-way and the two-way communication system for each of the chambers.

In other embodiments, the observer is located in a facility that is remote from one or more the chambers in another facility. The control system between the two facilities allows the observer to communicate with the user using a one-way communication or two-way communication between the observer and the user(s) in the one or more chambers in real time. In some embodiments, the observer can choose between the one-way and the two-way communication system.

In another embodiment, the observer is located in a facility that is remote from two or more chambers in two or more facilities, which are also remote from each other. The control system allows the observer to communicate with the two or more users in the two or more chambers located in the two or more remote facilities utilizing one-way or two-way communication with each other in real time. In some embodiments, the observer can choose between the one-way and the two-way communication system.

The control system also includes a monitoring and control capabilities where the system can monitor environmental conditions within the chamber and/or physiological data of the one or more users while the chamber is heated with infrared heaters or cooled with chilled air. The observer can utilize the collected environmental and physiological and/or psychological data to optimize the user's benefits from activities being conducted within the chamber by changing environmental conditions. By way of non-limiting example, if the user's internal body temperature is overly elevated, the observer can cause the temperature within the chamber to decrease by reducing the heat input or causing chilled air to be forced into the chamber. Alternatively, if the user is not achieving the desired effects of the activity in the chamber, the temperature can be increased by changing the heat output from the heaters, bringing additional heaters on-line or forcing hot air into the chamber. Also, by way of non-limiting example, if the blood oxygen content of the user is too low, oxygen enriched air can be directed into the chamber to address the blood oxygen content of the user. Conversely, the oxygen concentration in the chamber can be reduced to simulate altitude for oxygen acclimation. In other embodiment, air ionizers and/or air purifiers can be manipulated by the control system. In yet other embodiments, a humidity/steam system or an aromatherapy system can be manipulated by the control system to change the environmental conditions within the chamber. In yet other embodiments, visible light, such as but not limited to, controlled LED light, and/or electromagnetic energy, such as but not limited to, pulsed electromagnetic energy, can be manipulated by the control system to change the environmental conditions within the chamber. It is within the scope of the present disclosure to adjust any of the environmental variables individually or in combination with other environmental variables based upon the environmental and/or physiological data to enhance the user's experience.

The present disclosure utilizes a chamber that has one or more infrared heaters that are used to heat the chamber to the selected elevated temperature, where the one or more infrared heaters emit photons. The one or more infrared heaters can emit far infrared (FIR) wavelengths, mid infrared (MIR) wavelengths and/or near infrared (NIR) wavelengths to provide different effects to the user. By way of example FIR wavelengths penetrate deeper into the body to increase core temperature while NIR wavelengths provide topical heat to the skin of the user. MIR wavelengths provide both some surface heating and some penetration into the body. In some embodiments, the infrared heaters only emit FIR wavelengths. The one or more infrared heaters can also be controlled with the control system.

However, most electrical equipment such as screens, speakers, cameras and/or microphones cannot withstand the elevated temperatures for extended periods of time. Elevated temperatures include temperatures above 130° F. The present disclosure is configured to allow the electronic equipment to be used for extended periods of time within chambers at elevated temperatures by preventing the equipment from being fully exposed to the elevated temperatures while allowing the user or user to utilize the electronic equipment. By way of non-limiting example, a video display screen can be positioned behind a tempered glass pane that allows the screen to be viewed while in the chamber without exposing the screen to excessive temperatures. Also, a compact electronic device, such as a cellular telephone or an electronic tablet, can be stored in a recess that is protected from the elevated temperatures by a movable cover, such as tempered glass, such that the user can access the electronic device when desired and then the device can be placed back into the recess to protect the device from the elevated temperatures.

A camera may be installed behind protective material, such as tempered glass, such that sensitive electronic equipment is not exposed to elevated temperatures. Finally, a speaker and/or a microphone can optionally be installed where sound can be transmitted around or through a protective barrier so that the user can hear instructions from the observer and the user can also communicate with the observer. However, in some embodiments, the speaker is able to withstand the elevated environmental conditions of the chamber.

In another embodiment, the electronic equipment within the environmental chamber can include a commercially available electronic device, such as a smart television that includes at least a camera, a microphone and optionally a speaker, and a screen capable of receiving a video stream and a speaker for transmitting an audio feed. In other embodiments a hand held device, such as a cell phone or a tablet, can be sued to provide the camera, speaker, screen and microphone where the hand held device streams to a television located behind the tempered glass. In one embodiment, the hand held electronic device and the screen are hardwired into the control system. In other embodiments, the hand held electronic device and the screen are Wi-Fi enabled to communicate with the control system wirelessly.

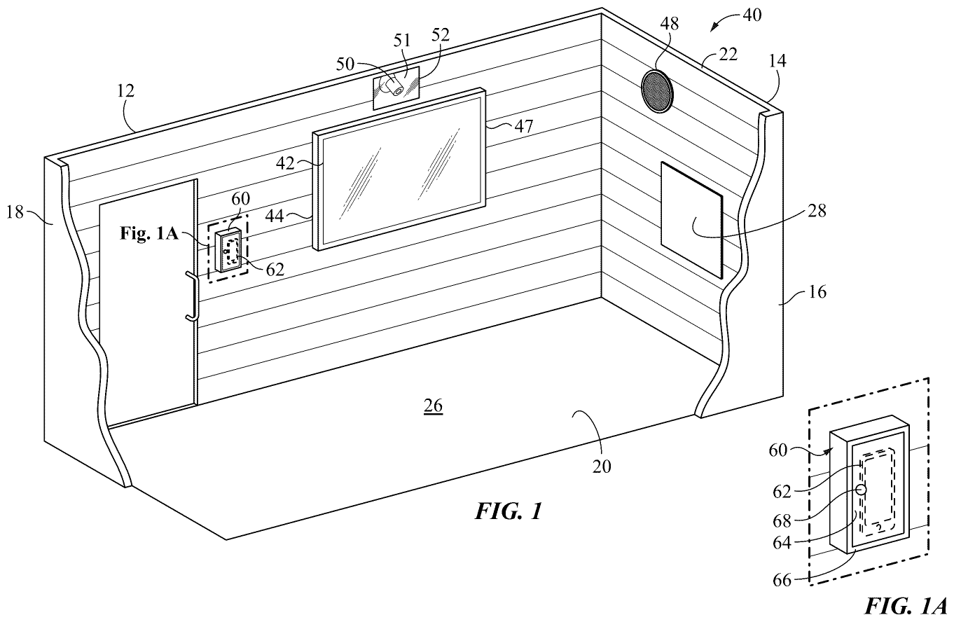

Referring to FIG. 1, a chamber 10 includes a left wall 12, a back wall 14, a right wall 16 and a front wall 18. The chamber 10 includes a floor 20 that attaches to the bottom edges of the walls 12-18 and a ceiling 22 that attaches to the top edges of the walls 12-18. The chamber 10 includes a door 24 in the left wall 18 that allows for entry and egress from an interior space 26 within the chamber 10. The chamber 10 includes at least one infrared heater 28 that is used to heat the interior space 26 to the selected elevated temperature. Typically, the at least one infrared heater 28 includes a far infrared spectrum (FIR) heater having wavelength ranges from about 5,600 nm to about 100,000 nm. Typically, the chamber 10 includes more than one IR heater 28 to aid in heating the interior space 26. By way of non-limiting example, a chamber with multiple IR heaters is disclosed and illustrated in Daffer U.S. Printed Application Publication No. 2020/0384288A1, which is incorporated by reference herein that includes MIR and NIR heaters. The present disclosure can be utilized in the photonic chamber Daffer U.S. Printed Application Publication No. 2020/0384288A1 with the addition of the control system and its components and optionally removing the bench and to provide an even floor without a bench or heater. The interior space 26 is sized to allow any number of people to exercise, and is typically sized to allow 1 to 4 people to exercise therein.

The chamber 10 includes components of a communication system within a control system 40. The components include a camera 50 is positioned through an aperture 52 in the left wall 12 where the camera 50 is at a height that provides a view of person while exercising. In some instances, the camera 50 is capable of tracking movement. However, the aperture 52 can be located in any of the other walls. The aperture 52 for the camera 50 can be covered with a substantially transparent, insulating material to protect the camera 50 from the elevated temperature within the chamber 10. A typical, non-limiting, substantially transparent, insulating material includes a pane of tempered glass 51.

The communication system within the control system 40 also includes one or more speakers 48 that are located in the back wall 14. However, the speakers 48 can be located within any of the walls. The one or more speakers 48 are configured to receive signals either through a hard wire or wirelessly such that the audio stream from the observer can be heard. The camera 50 and the one or more speakers 48 are used for one-way communication from the observer to the user.

The communication system within the control system 40 includes a screen 42 within a recess or cut out 44 within the left wall 46 that is covered by a pane of tempered glass 47 to shield the screen 42 from the elevated temperatures within the interior space 26. The screen 42 is configured to receive signals either through a hard wire or wirelessly from an external router such that the observer can be viewed by the user. However, the recess or cut out 44 can be located in any of the other walls.

The camera 50 can be hard wired or wirelessly connected to the communication system within the control system 40 such that a stream of the user can be viewed by a third party, such as the observer. The observer can the communicate with the user(s) via the speakers 48. The system optionally includes a microphone 51 that is configured to send an audible stream from the user to the observer or trainer.

Referring to FIGS. 1 and 1A, the chamber 10 can also optionally include a recess 60 sized to accept a hand held device, such as, but not limited to a cellular telephone 62. The opening 64 to the recess is covered with a door 66 having an insulating material, such as at least partially a pane of tempered glass. A handle 68 is attached to the door 66 to move the door 66 from a first position covering the recess to a second position to provide access to the recess 60, which also allows the user to protect the hand held communication device from excessive heat for extended periods of time when positioned in the recess 60 and the door 66 covers the recess 60. When in the door 66 is in the second open position, the hand held device 62 can be retrieved and used for a short period of time with the chamber 10 at the elevated temperature. Once the use is completed, the hand held device 62 is placed into the recess 60 and the door 66 is placed into the first position to protect the hand held device from the elevated temperatures. The door can be positioned from the first position to the second position and back using a connecting member, such as a hinge, a slide and/or a spring biased hinge or slide. In some embodiments, the screen 42 is configured to receive and display stream directly from the hand held device 62 such that audio and video streams received by the hand held device 62 are received and displayed or able to be heard through the screen 42.

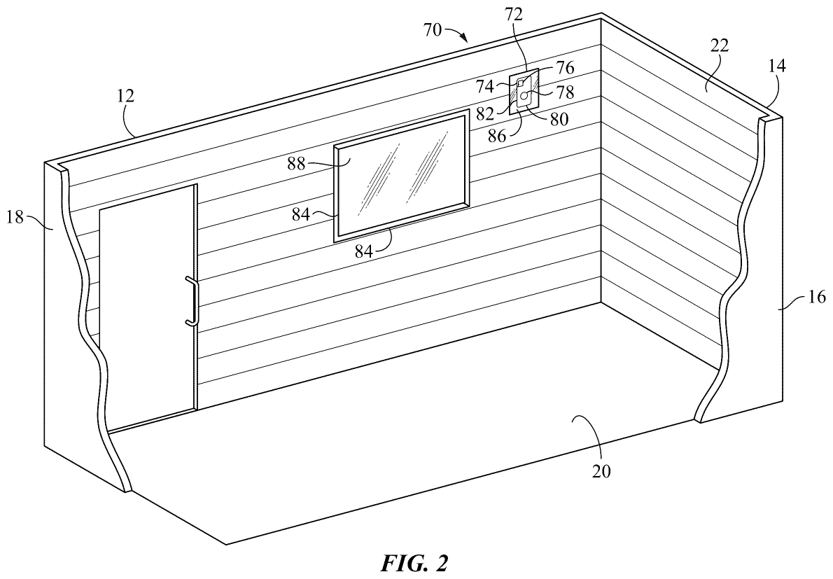

Referring to FIG. 2, another embodiment of the communications system within the control system within the chamber 10 is illustrated at 70. The communication system within the control system 70 includes a recess 72 that is configured to accept a hand held electronic device 74 that includes a camera 76 configured to record the user and a speaker 78 configured to provide audible communication to the user from the observer. The device 74 can include a microphone 80 and a screen 82 where the microphone 80 allows the user to communicate with the observer and the screen 82 can be streamed to a larger screen 84 located within another recess 85. Alternatively, the larger screen 84 can receive a video stream for the control system 70. Both the larger screen 84 and the hand held communication device 74 are protected with an insulating and substantially optically transparent material such as, but not limited to tempered glass 86 and 88. In some instances, the recesses include openings to cause convective cooling. In other instances, cooling fluid, such as air is forced through the recess 72 and 85 a to actively cool the larger screen 84 and/or the hand held electronic device 74.

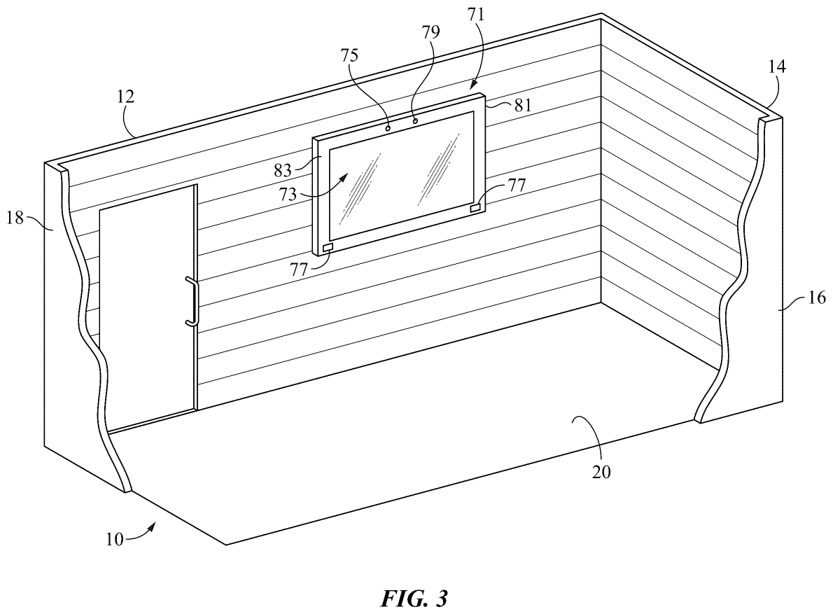

Referring to FIG. 3, another embodiment of embodiment of a communication system within a control system illustrated within the chamber is illustrated at 71. Instead of having multiple components. The communication system with the control system 71 includes a smart television with a screen 73, a camera 75, speakers 77 and a microphone 79. The smart television is located within a recess 81 that is covered by tempered glass 83 to protect the smart television from excessive heat.

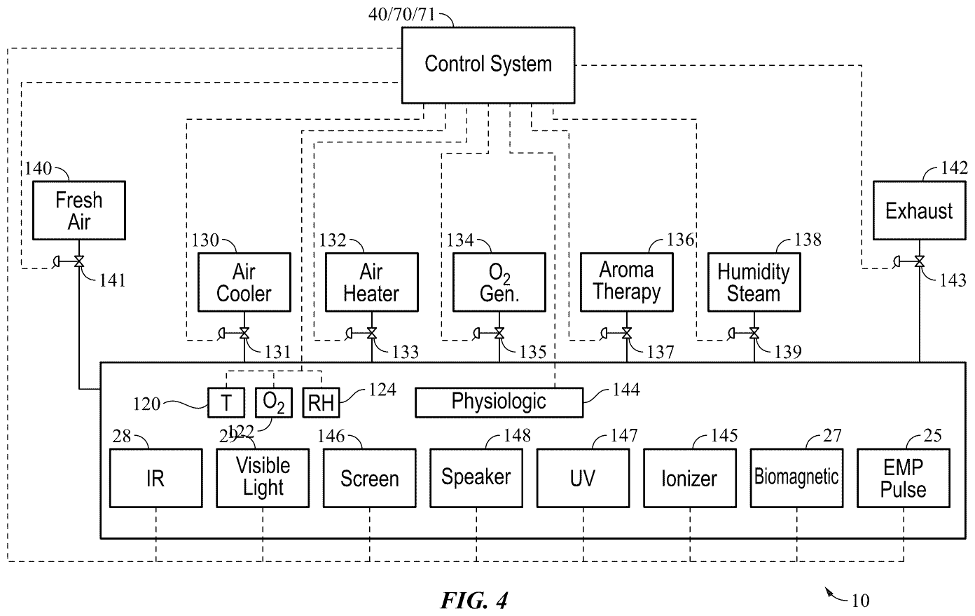

Referring to FIG. 4, a schematic view of the chamber 10 with the environmental sensors within the chamber, physiological sensors and external environmental modalities used to manipulate the environment are illustrated. The chamber 10 includes the one or more IR heaters 28 and chromatic visible lights 29, such as LED light arrays as disclosed in Daffer U.S. Printed Application Publication No. 2020/0384288A1. The chamber also includes a temperature sensor 120 to determine the temperature in the chamber, an optional an oxygen sensor 122 to determine oxygen concentration in the chamber and an optional hygrometer 124 to measure humidity in the chamber. The chamber 10 can include biomagnetic emitting materials 27, such as but not limited to steel, cobalt and nickel, and/or electromagnetic pulse engines 25 configured to emit pulses of electromagnetic energy.

External environmental modalities include an air cooler or chiller 130, an air heater 132, an oxygen generator 134, an aroma therapy nebulizer or dispenser 136 and/or a humidity or steam generator 138. The environmental modalities also include a fresh air intake 140 and an exhaust system 142. Internal modalities within the chamber include an air ionizer 145 and/or an air disinfectant 147, such as a UV light source.

The user is in communication with at least one wearable device 144 that measure one or more of the physiological data. The one or more wearable devices 144 and the temperature sensor 120 and optional oxygen sensor 122 and hygrometer 124 are in communication with the control system 40, 70 and/or 71 such that the observer can monitor the environmental conditions within the chamber 10 and the physiological data of the user.

Based upon the analyzed data from one or more of the wearable device 144, the temperature sensor 120, the oxygen sensor 122 and/or the hygrometer 124, the user can manipulate of the modalities by either energizing the modality 130, 132, 134, 136, 138, 140, 142, 145 and/or 147 manipulating control valve 131, 133, 135, 137, 139, 141 and 143 for the modalities 130, 132, 134, 136, 138, 140 and/or 142, all respectively.

By way of example if a user's internal body temperature is below a predetermined range for optimal hypothermic conditioning, the air heater 132 may be energized and the control valve 133 may be opened using the control system 40, 70, 71 to force hot air into the chamber 10 to quickly raise the environmental temperature within the chamber 10, which in turn should raise the user's internal body temperature. Conversely, if the user's internal body temperature is too high, the observer can energize the air chiller 130 and/or open the control valve 131 using the control system 40, 70, 71 to quickly lower the environmental temperature within chamber 10, which should lower the user's internal body temperature.

If the user's VO_{2 max} is sensed as declining, the observer can energize the concentrated oxygen generator 134 and/or open the control valve 135 using the control system 40, 70, 71 to increase the oxygen concentration in the chamber to stabilize or increase the user's VO_{2 max}. If the user is conditioning for exertion in a humid climate, the observer can raise the humidity level within the chamber 10, by energizing that humidity or steam generator 138 and/or opening the control valve 138 using the control system 40, 70, 71. When nebulizing of a topical liquid, such as a liquid vitamin and/or cosmenutraceutical and/or aroma therapy is desired in the chamber, the observer can energize a nebulizer and/or the aroma therapy dispenser 136 and/or open the control valve 137 using the control system 40, 70, 71. When the observer desires to change out the air in the chamber 10, the air intake 140 can be energized and/or the control valve 141 and the exhaust 142 can be energized and/or the control valve opened using the control system 40, 70, 71 to change out the air in the chamber 10.

When the user's activity requires a screen 146, the observer can control the screen 146 with the control system 40, 70, 71 and can provide information, such as input from a trainer when exercising, or calming visual effects, such as when meditating. Similarly, observer can control the speaker 148 with the control system 40, 70, 71 to provide information and/or calming sounds.

The observer can control the modalities by themselves or in combination with one another to adjust the environmental conditions in the chamber 10, based upon and sensed condition, physiological data and/or time. The combinations of modalities modifications to the chamber 10 allows the user to maximize the beneficial effects of the activity in the chamber 10.

Once, the user is finished in the chamber 10, the UV lights 147 can be energized to emit UVC lights that is used to cleanse and sterilize the chamber air and surfaces. The UVC lights can aid in providing a more clean and sterile environments between sessions by a user or by different users using the same chamber 10.

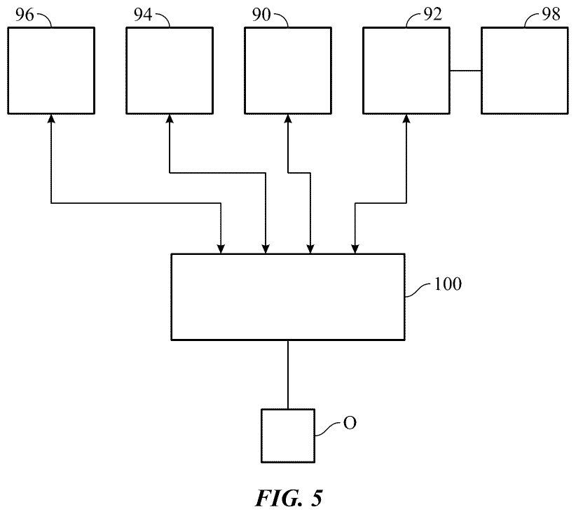

Referring to FIG. 5, the observer O can have additional equipment of the control system 40, 70 and 71 where the observer can have a screen 90 configured to receive the video stream from the camera 50 or 76 and a microphone 92 configured to send an audible stream to the speaker 48 or 78. This configuration provides for one-way communication from the observer with the user.

The observer's equipment can include a camera 94 configured to stream video to the screen 42, 73 and 82/84 and a speaker 96 configured to receive an audible stream from the microphone 51, 79 or 80. The use of the screen 90, the microphone 92, the camera 94 and the speaker 96 with the electrical components in the communication system 40, 70 and 71 allows for two-way communication between a person a chamber and the observer or trainer. In many instances when the observer is working with multiple chambers with multiple users, the control system 40, 70 and 71 includes a control feature 96 that allows the observer to switch between one-way and two-way communication, so that the observer can monitor all chambers with the users while directly communicating with a single chamber. Further, the observer also has a control feature 98 to only turn the audio feed from the microphone 92 to a single chamber.

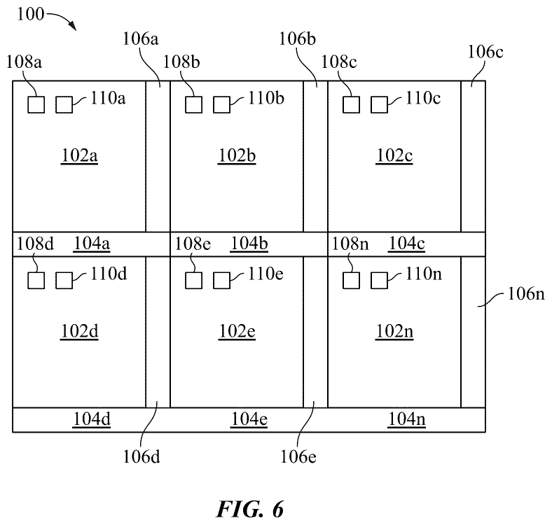

Referring to FIG. 6, an exemplary graphic user interface (GUI) on the screen that is either a touch screen or controlled by a keyboard and mouse is illustrated at 100. The GUI 100 include one or more split screen displays 102ₐ-102n where n is the number of chambers being monitored. Each split screen display 102ₐ-102n includes a display of the monitored or sensed physiological data from devices worn by the user at 104ₐ-104n located within each split screen 102ₐ-102n. Each split screen 102ₐ-102n includes a display of environmental conditions 106ₐ-106n located within each split screen 102ₐ-102n. Each split screen 102ₐ-102n includes a display 108ₐ-108n to activate an audio stream from the observer to the selected chamber such that one-way or two-way audio communication can be established with one or more selected chambers. Each split screen 102ₐ-102n also include a display 110ₐ-110n to activate and deactivate a video stream of the observer to the user in a selected chamber.

In some embodiments upper and lower limits are provided to the system for the physiological or psychological data. When the monitored or sensed physiological or psychological data moves beyond the upper or lower limit, the GUI provides an indication to the observer that attention is required. In some instances, the observer will open a one-way or two-way communication to determine the cause of the indication and/or result in the observer adjusting one or more environmental conditions through the GUI 100.

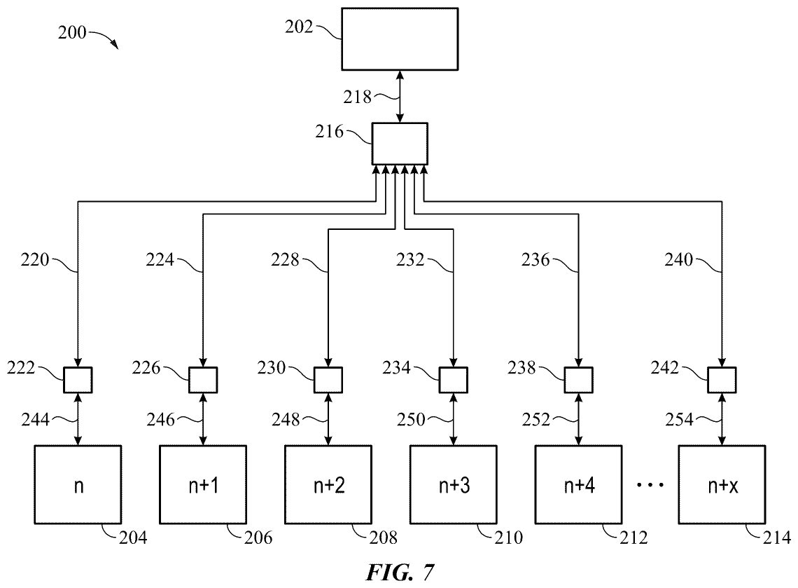

Referring to FIG. 7, a control system is illustrated at 200 that is contained within a single facility where the control system is configurable to be one way communication, two way communication or a modified two way communication where video is streamed and audio feedback is only provided to selected chambers. The control system 200 as illustrated is configured to allow an observer to be located in a location 202 remote from one or more selected chambers. In some embodiments, the location 202 is a heated chamber and in other embodiments the location 202 is in ambient conditions. As illustrated, the one or more heated chambers include six chambers 204, 206, 208, 210, 212 and 214. Chamber 204 is designated as n, and all subsequent chambers 206, 208, 210, 212 and 214 are designated with the next consecutive number. It is noted that chamber 214 is designated as n+x, meaning the control system 200 can be utilized with any of a number of chambers and is not limited to the illustrated six chambers.

Typically, each of the chambers 202-214 are similarly equipped with a screen, camera, speakers, microphone, and a control panel and an optional a recess for storing hand-held electronic equipment. The components of the electronic equipment can be separate or can be included in one or more devices, such as a cellular telephone having all four pieces of equipment and or a television having a screen and a speaker. Having the chambers 202-214 being similarly equipped allows the chambers to be interchangeable.

The observer is in a location 202 that is remote from the chambers but in the same facility where the observer or trainer can monitor each chamber 202-214 on the screen 90. The observer receives at least a video stream from one or more of the chambers 202-214 along with the physiological data and/or environmental conditions. The observer can then provide audible feedback with the microphone 92 to one or more of the chambers 202-214 using the control 98 to provide one-way communication. The observer in the location 202 can also enable the camera 94 and the speaker 96 so that one or more chambers 202-214 has two-way communication with the observer.

The control system 200 includes a server 216 that receives signals from one or more of the observer's microphone 92, camera 94 and speaker 96 then transmits signals from the observer to receiver/transmitters 222, 226, 230, 234, 238 and 242 along hard wired or wireless transmission lines 220, 224, 228, 232, 236 and 240, respectively. The receiver/transmitters 222, 226, 230, 234, 238 and 242 then transmit signals relating to video and audible stream sent by the observer to the screen and speakers in selected of the chambers 204, 206, 208, 210, 212 and 214 along hard wired or wireless transmission lines 244, 246, 248, 250, 252 and 254, respectively.

Similarly, the wearable devices and sensors in the chambers sends signals through the transmission lines 244, 246, 248, 250, 252 and 254, through the receiver/transmitters 222, 226, 230, 234, 238 and 242, across transmission lines 220, 224, 228, 232, 236 and 240 to the server 216 and finally to the GUI 100. Additionally, the observer can manipulate one or more of the modalities 130, 132, 134, 136, 138, 140, 142, 145 and/or 147 based upon the sensed physiological data or the environmental conditions.

Additionally, the observer can optionally transmit video of the observer that is visible on the screens of the selected chambers 202-214. The observer can also optionally enable a microphone in a chamber 202-214 such that the user can communicate with the observer.

As illustrated and described, each of the chambers 204-214 is being utilized. However, if one or more chambers 204-214 is not being utilized, then the system 200 is configured to turn off the communication lines to the unused chamber.

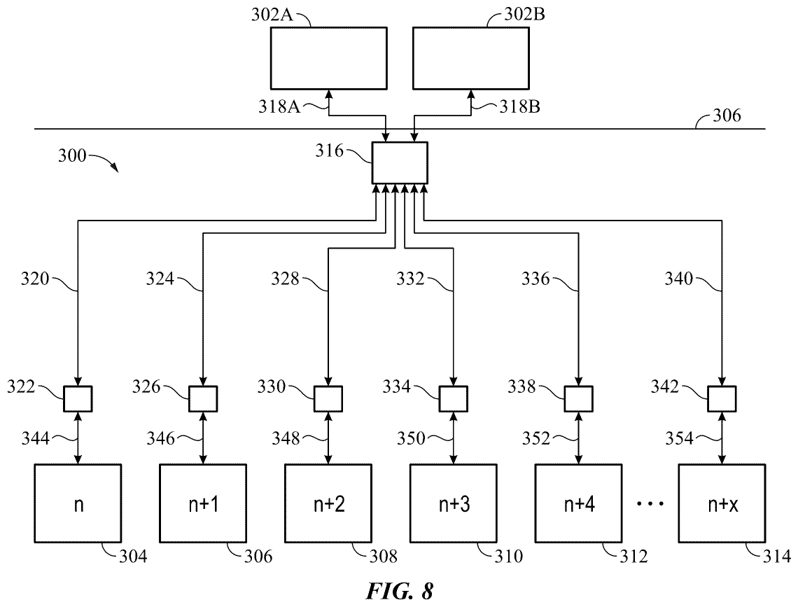

Referring to FIG. 8, another control system is illustrated at 300. The control system 300 includes similar features as that of the control system 200, besides being in different facilities as illustrated by the line 306. The control system 300 also includes the concept of multiple observers 302S and 302B be able both monitor and control the chambers 302-314 or to divide responsibilities for the chambers.

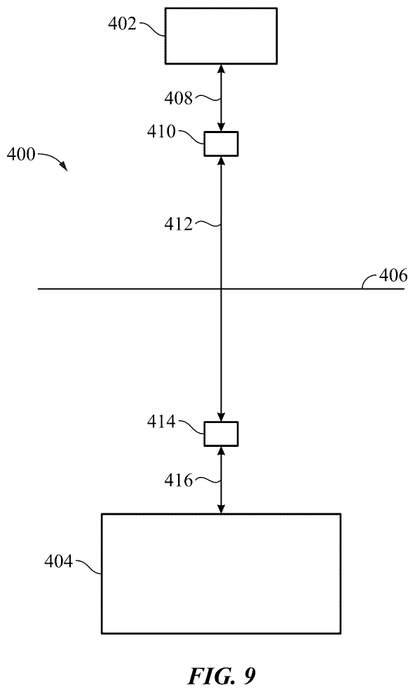

Referring to FIG. 9, another control system is illustrated at 400. The system 400 allows the observer 402 to interact with a user in a chamber 404 at the user's residence. In some embodiments the observer 402 can be located in a different facility relative to the facility where the chamber 404 is located as illustrated by the line 406. However, the observer 402 can also be located in the user's residence at the time the user is in the chamber 404.

The chamber 404 includes the screen, speakers, microphone and camera as previously discussed. The observer 402 can also be recorded by a camera such that signals relating to the visual and audio recorded by the camera is transmitted along a hard wired or wireless communication line 408 to a receiver/server 410. The receiver/server 410 sends the signals along a hard wired or wireless communication line 412 to a receiver/router 414 that in turn sends the signal along a hard wired or wireless communication line 416 to the chamber 404, where the instructor/trainer 402 is streamed to the screen and the audio can be heard through the speakers. The control system 400 also transmits physiological and/or psychological data and environmental conditions that can be monitored by the observer where the observer can optionally manipulate the environmental conditions based upon the physiological and/or psychological data.

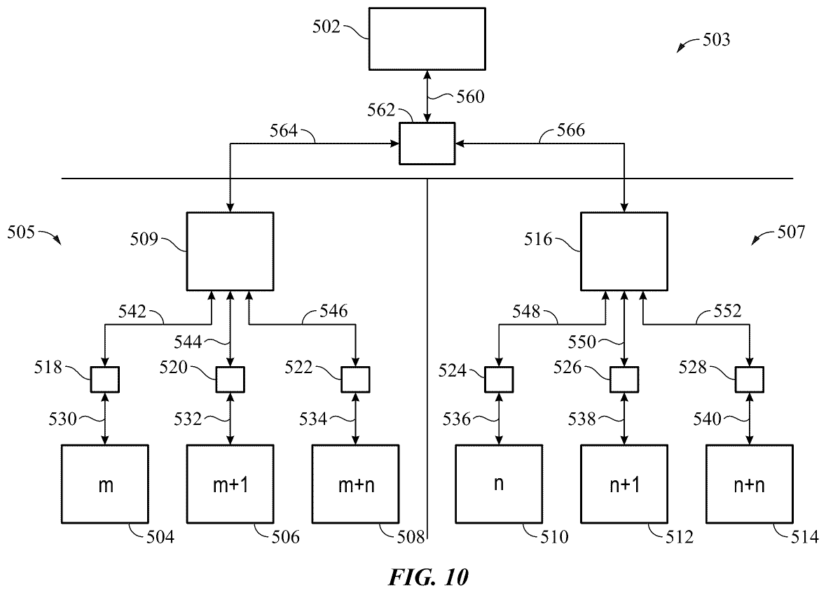

Referring to FIG. 10, another control system is illustrated at 500. The control system 500 allows an observer 502 in one facility monitor 503, communicate with and control environmental conditions in chambers 5 in a second facility 505 and chambers n . . . n+n in a third facility 507. The number of chambers and number of facilities that can be observed and controlled by the observer 502 can be any selected number, including greater than two.

The chambers 504. 506, 508, 510, 512, 514 includes the screen, speakers, microphone and camera as previously discussed. The observer 502 is also being recorded by a camera such that signals relating to the visual and audio recorded by the camera is transmitted along a hard wired or wireless communication line 560 to a receiver/server 562. The receiver/server 562 sends the signals along a hard wired or wireless communication line 564 and 566 to a receiver/router 509 in the facility 505 and the receiver/router 516 in the facility 507 that in turn sends the signal along a hard wired or wireless communication lines 542, 544, 546, 548, 550 and 552 to the chambers 504. 506, 508, 510, 512, 514 through routers/receivers 518, 520, 522, 524, 526 and 528 and lines 530, 532, 534, 536, 538 and 540 all respectively. The control system 50 also transmits physiological and/or psychological data and environmental conditions that can be monitored by the observer where the observer can optionally manipulate the environmental conditions based upon the physiological and/or psychological data.

Although the present disclosure has been described with reference to preferred embodiments, workers skilled in the art will recognize that changes may be made in form and detail without departing from the spirit and scope of the disclosure.

1. A method of controlling an environmentally controlled chamber, the method comprising:
providing a chamber with at least one infrared heater, a screen configured to stream content, the screen positioned behind an insulating barrier comprising a substantially optically transparent portion, a camera configured to record a user and speakers within the chamber;
sending the user's physiological and/or psychological data and environmental conditions within the chamber to a location remote from the chamber for monitoring; and
changing environmental conditions within the chamber based upon the received physiological and/or psychological data or sensed environmental conditions from the location remote from the chamber.

2. The method of claim 1, and further comprising:
receiving feedback or instructions over the speakers for the user while within the chamber through the speaker. 3, The method of claim 1, and further comprising:
streaming video content of an observer spaced from the chamber onto the screen for the user to view in real time.

4. The method of claim 1, and further comprising:
streaming audio from the microphone to an observer spaced from the chamber such that the observer can receive audio from the user in real time.

5. The method of claim 1, wherein controlling the environmental conditions comprises:
adjusting a temperature within the chamber based upon the user's body temperature.

6. The method of claim 5, wherein adjusting the temperature comprises:
causing chilled air to be directed into the chamber to lower the temperature within the chamber.

7. The method of claim 5, wherein adjusting the temperature comprises:
causing heated air to be directed into the chamber to raise the temperature within the chamber.

8. The method of claim 1, wherein controlling the environmental conditions comprises:
adjusting oxygen content within the chamber based upon the user's maximum rate of oxygen consumption.

9. The method of claim 5, wherein controlling the environmental conditions comprises:
manipulating a humidity or aroma in the chamber.

10. The method of claim 5, wherein controlling the environmental conditions comprises:
adjusting a delivery of electromagnetic radiation or visible light into the chamber.

11. The method of claim 1, wherein the chamber is provided with a recess in a wall with an insulated door, the method further comprising:
opening the door to provide access to the recess;
positioning a hand held electronic device with the chamber; and
closing the door to protect the hand held device from the environmental conditions within the chamber.

12. A method of controlling a plurality of environmentally controlled chambers, the method comprising:
providing a plurality of spaced apart chamber with at least one infrared heater, a screen configured to stream content, the screen positioned behind an insulating barrier comprising a substantially optically transparent portion, a camera configured to record a user and speakers within the chamber;
sending the user's physiological and/or psychological data and environmental conditions within each of the plurality of chambers to a location remote from the chamber for monitoring; and
changing environmental conditions within one of the plurality of chambers based upon the received physiological and/or psychological data or sensed environmental conditions within the one chamber of the plurality of chambers based upon signals sent from the location remote from the one chamber of the plurality of chambers.

13. The method of claim 12, and further comprising:
receiving feedback or instructions over the speakers for the user while within one of the plurality of chambers through the speaker.

14. The method of claim 12, and further comprising;
streaming video content of an observer spaced from the plurality of chambers onto the screen of at least one of the plurality of chambers for the user to view in real time.

15. The method of claim 12, and further comprising:
streaming audio from the microphone to an observer spaced from the chamber such that the observer can receive audio from the user in real time.

16. The method of claim 12, wherein controlling the environmental conditions within one of the plurality of chambers comprises:
adjusting a temperature within the one chamber of the plurality of chambers based upon the user's body temperature.

17. The method of claim 16, wherein adjusting the temperature comprises:
causing chilled air to be directed into the chamber to lower the temperature within the chamber.

18. The method of claim 16, wherein adjusting the temperature comprises:
causing heated air to be directed into the chamber to raise the temperature within the chamber.

19. The method of claim 12, wherein controlling the environmental conditions within one of the plurality of chambers comprises:
adjusting oxygen content within the chamber based upon the user's maximum rate of oxygen consumption.

20. The method of claim 12, wherein controlling the environmental conditions within one of the plurality of chambers comprises:
adjusting a delivery of electromagnetic radiation or visible light into the chamber.
